(19) United States
(12) Patent Application Publication
(10) Pub. No.: US 2005/0266536 A1
(43) Pub. Date: Dec. 1, 2005

(54) Process for the fermentative preparation of L-amino acids using coryneform bacteria

(75) Inventors: Andreas Wolf, Koeln (DE); Natalie Schischka, Bielefeld (DE); Thomas Hermann, Bielefeld (DE); Susanne Morbach, Juelich (DE); Reinhard Kraemer, Juelich (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Duesseldorf (DE)

(21) Appl. No.: 11/124,291

(22) Filed: May 9, 2005

Related U.S. Application Data
(62) Division of application No. 10/212,219, filed on Aug. 6, 2002.
(60) Provisional application No. 60/316,276, filed on Sep. 4, 2001.

(30) Foreign Application Priority Data
Aug. 9, 2001 (DE) 101 39 062.9

(51) Int. Cl. C12P 13/04; C12P 13/14; C07H 21/04; C12P 21/06; C12N 9/16

(52) U.S. Cl. 435/110; 435/69.1; 435/196; 435/252.3; 435/471; 536/23.2; 435/115

(57) ABSTRACT

A process for the preparation of L-amino acids, in which the following steps are carried out: (a) fermentation of the coryneform bacteria which produce the desired L-amino acid and in which at least the gene which codes for trehalose phosphatase and/or the gene which codes for maltooligosyl-trehalose synthase and/or the gene which codes for maltooligosyl-trehalose trehalohydrolase is or are attenuated, (b) concentration of the desired L-amino acid in the medium or in the cells of the bacteria, and (c) isolation of the L-amino acid, and optionally bacteria in which further genes of the biosynthesis pathway of the desired L-amino acid are addtionally enhanced are employed, or bacteria in which the metabolic pathways which reduce the formation of the desired L-amino acid are at least partly eliminated are employed.

### CROSS REFERENCE TO RELATED APPLICATIONS

This application claims benefit to U.S. Provisional Application Ser. No. 60/316,276, filed on Sep. 4, 2001, and to German Patent Application Ser. No. 101 39 062.9, filed on Aug. 9, 2001, both of which are incorporated herein by reference.

### BACKGROUND OF THE INVENTION

1. Field of the Invention

The present invention relates to a process for the fermentative preparation of L-amino acids, in particular L-lysine and L-glutamic acid, using coryneform bacteria in which one or more genes chosen from the group consisting of the otsB gene, treY gene and treZ gene are attenuated.

2. Description of the Background

L-Amino acids, in particular L-lysine and L-glutamic acid, are used in human medicine and in the pharmaceuticals industry, in the foodstuffs industry and very particularly in animal nutrition.

It is known that amino acids are prepared by fermentation from strains of coryneform bacteria, in particular Corynebacterium glutamicum. Because of their great importance, work is constantly being undertaken to improve the preparation processes. Improvements to the process can relate to fermentation measures, such as, for example, stirring and supply of oxygen, or the composition of the nutrient media, such as, for example, the sugar concentration during the fermentation, or the working up to the product form by, for example, ion exchange chromatography, or the intrinsic output properties of the microorganism itself.

Methods of mutagenesis, selection and mutant selection are used to improve the output properties of these microorganisms. Strains which are resistant to antimetabolites, such as, for example, the lysine analogue S-(2-aminoethyl)-cysteine, or are auxotrophic for metabolites of regulatory importance and produce L-amino acids are obtained in this manner.

Methods of the recombinant DNA technique have also been employed for some years for improving the strain of Corynebacterium glutamicum strains which produce L-amino acids, by amplifying individual amino acid biosynthesis genes and investigating the effect on the L-amino acid production.

### SUMMARY OF THE INVENTION

The inventors had the object of providing new fundamentals for improved processes for the fermentative preparation of L-amino acids, in particular L-lysine and L-glutamic acid, with coryneform bacteria.

It is another object of the invention to provide nucleotide sequences which may be used to accomplish this object.

The objects of the invention may be accomplished with an isolated polynucleotide from coryneform bacteria, containing a polynucleotide sequence which codes for trehalose phosphatase and/or a polynucleotide sequence which codes for maltooligosyl-trehalose synthase and/or a polynucleotide sequence which codes for maltooligosyl-trehalose trehalohydrolase, wherein each sequence is lengthened by approximately 600 base pairs before the start codon and after the stop codon.

The invention provides also a process for the fermentative preparation of L-amino acids using coryneform bacteria in which at least the nucleotide sequence which codes for trehalose phosphatase and/or the nucleotide sequence which codes for maltooligosyl-trehalose synthase and/or the nucleotide sequence which codes for maltooligosyl-trehalose trehalohydrolase is or are attenuated, in particular eliminated or expressed at a low level.

The present invention also provides a process for the fermentative preparation of L-amino acids, in which the following steps are carried out:
(a) fermentation of the L-amino acid-producing coryneform bacteria in which at least the nucleotide sequence which codes for trehalose phosphatase and/or the nucleotide sequence which codes for maltooligosyl-trehalose synthase and/or the nucleotide sequence which codes for maltooligosyl-trehalose trehalohydrolase is or are attenuated, in particular eliminated or expressed at a low level;
(b) concentration of the L-amino acids in the medium or in the cells of the bacteria; and
(c) isolation of the desired L-amino acids, constituents of the fermentation broth and/or the biomass optionally remaining in portions or in their total amounts in the end product.

A more complete appreciation of the invention and many of the attendant advantages thereof will be readily obtained as the same becomes better understood by reference to the following Figures in conjunction with the detailed description below.

### BRIEF DESCRIPTION OF THE FIGURES

FIG. 1: Map of the plasmid pCR2.1otsBint,

FIG. 2: Map of the plasmid pCR2.1treYint,

FIG. 3: Map of the plasmid pCR2.1treZint.

The abbreviations and designations used have the following meaning.

KmR: Kanamycin resistance gene

BamHI: Cleavage site of the restriction enzyme KpnI

EcoRI: Cleavage site of the restriction enzyme EcoRI

EcoRV: Cleavage site of the restriction enzyme EcoRV

PstI: Cleavage site of the restriction enzyme PstI

SalI: Cleavage site of the restriction enzyme SalI

otsBint: Internal fragment of the otsB gene

treYint: Internal fragment of the treY gene

treZint: Internal fragment of the treZ gene

ColE1: Replication origin of the plasmid ColE1

### DETAILED DESCRIPTION OF THE INVENTION

Where L-amino acids or amino acids are mentioned in the following, this means one or more amino acids, including their salts, chosen from the group consisting of L-asparagine, L-threonine, L-serine, L-glutamic acid, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-Lysine and L-glutamic acid are particularly preferred.

When L-lysine or lysine are mentioned in the following, not only the bases but also the salts, such as e.g. lysine monohydrochloride or lysine sulfate, are meant by this.

When L-glutamic acid or glutamic acid are mentioned in the following, the salts, such as e.g. glutamic acid hydrochloride or glutamic acid sulfate are also meant by this.

The strains employed preferably already produce L-amino acids, in particular L-lysine and L-glutamic acid, before the attenuation of the otsB gene, which codes for trehalose phosphatase, and/or the treY gene, which codes for maltooligosyl-trehalose synthase, and/or the treZ gene, which codes for maltooligosyl-trehalose trehalohydrolase.

The term “attenuation” in this connection describes the reduction or elimination of the intracellular activity of one or more enzymes (proteins) in a microorganism which are coded by the corresponding DNA, for example by using a weak promoter or using a gene or allele which codes for a corresponding enzyme with a low activity or inactivates the corresponding gene or enzyme (protein), and optionally combining these measures.

By attenuation measures, the activity or concentration of the corresponding protein is in general reduced to 0 to 75%, 0 to 50%, 0 to 25%, 0 to 10% or 0 to 5% of the activity or concentration of the wild-type protein or of the activity or concentration of the protein in the starting microorganism. These ranges include all specific values and subranges therebetween, such as 2, 3, 8, 12, 15, 20, 30, 40, 60, and 70% of the activity or concentration of the wild-type protein or of the activity or concentration of the protein in the starting microorganism.

The microorganisms provided by the present invention can prepare amino acids from glucose, sucrose, lactose, fructose, maltose, molasses, starch, cellulose or from glycerol and ethanol. They can be representatives of coryneform bacteria, in particular of the genus Corynebacterium. Of the genus Corynebacterium, there may be mentioned in particular the species Corynebacterium glutamicum, which is known among experts for its ability to produce L-amino acids.

Suitable strains of the genus Corynebacterium, in particular of the species Corynebacterium glutamicum, are in particular the known wild-type strains
Corynebacterium glutamicum ATCC13032
Corynebacterium acetoglutamicum ATCC15806
Corynebacterium acetoacidophilum ATCC13870
Corynebacterium melassecola ATCC 17965
Corynebacterium thermoaminogenes FERM BP-1539
Brevibacterium flavum ATCC14067
Brevibacterium lactofermentum ATCC13869 and
Brevibacterium divaricatum ATCC14020
   and L-amino acid-producing mutants or strains prepared therefrom such as, for example, the L-lysine-producing strains
Corynebacterium glutamicum FERM-P 1709
Brevibacterium flavum FERM-P 1708
Brevibacterium lactofermentum FERM-P 1712
Corynebacterium glutamicum FERM-P 6463
Corynebacterium glutamicum FERM-P 6464 and
Corynebacterium glutamicum DSM 5715.

It has been found that coryneform bacteria produce L-amino acids in an improved manner after attenuation of the otsB gene, which codes for trehalose phosphatase (EC:3.1.3.12), and/or the treY gene, which codes for maltooligosyl-trehalose synthase, and/or the treZ gene, which codes for maltooligosyl-trehalose trehalohydrolase.

The nucleotide sequence of the gene which codes for the trehalose phosphatase of Corynebacterium glutamicum can be found in the patent application WO 01/00843 under Identification Code RXA00347 as SEQ ID No. 1139.

The nucleotide sequence of the gene which codes for the maltooligosyl-trehalose synthase of Corynebacterium glutamicum can be found in the patent application WO 01/00843 under Identification Code FRXA01239 as SEQ ID No. 1143.

The nucleotide sequence of the gene which codes for the maltooligosyl-trehalose trehalohydrolase of Corynebacterium glutamicum can be found in the patent application WO 01/00843 under Identification Code RXA02645 as SEQ ID No. 1145.

The nucleotide sequences are also deposited in the gene library under Accession Number AX064857, AX064861 and AX064863.

The nucleotide sequences of the present invention, of the genes which code for trehalose phosphatase, for maltooligosyl-trehalose synthase and for maltooligosyl-trehalose trehalohydrolase, shown in SEQ ID No. 1, SEQ ID No. 3 or SEQ ID No. 5 are lengthened compared with the sequences known from the publications cited above by in each case preferably up to 700 base pairs before the start codon and after the stop codon of the gene.

The lengthenings compared with the sequence known from the publications cited above comprise base pairs 1 to 500 and 1392 to 1977 in SEQ ID No. 1.

In SEQ ID No. 3 the lengthenings compared with the sequence known from the publications cited above comprise base pairs 1 to 500 and 3057 to 3636.

In SEQ ID No. 5 the lengthenings compared with the sequence known from the publications cited above comprise base pairs 1 to 500 and 2454 to 3033.

The amino acid sequences of the associated gene products are shown in SEQ ID No. 2, SEQ ID No. 4 or SEQ ID No. 6.

It has been found that attenuation processes which are known per se can be employed particularly successfully with the aid of the lengthened sequences thus provided.

Such a process is the method of gene replacement. In this, a mutation, such as e.g. a deletion, insertion or base exchange, is established in vitro in the gene of interest. The allele prepared is in turn cloned in a vector which is not replicative for C. glutamicum and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, in EP: 00110021.3 to eliminate the secG gene of C. glutamicum.

The lengthening of the sequences employed is not limited to 600 base pairs before the start codon and after the stop codon. It is preferably in the range from 300 to 700 base pairs, but can also be up to 800 base pairs. These ranges include all specific values and subranges therebetween, such as 325, 350, 375, 400, 425, 450, 475, 500, 525, 550, 575, 600, 625, 650, 675, 700, 725, 750, and 775 base pairs. The lengthenings can also contain different amounts of base pairs.

The sequences described in the text references mentioned which code for trehalose phosphatase, maltooligosyl-trehalose synthase and maltooligosyl-trehalose trehalohydrolase can be used according to the invention. Alleles of trehalose phosphatase, maltooligosyl-trehalose synthase or maltooligosyl-trehalose trehalohydrolase which result from the degeneracy of the genetic code or due to “sense mutations” of neutral function can furthermore be used.

To achieve an attenuation, either the expression of the gene which codes for trehalose phosphatase and/or the expression of the gene which codes for maltooligosyl-trehalose synthase and/or the expression of the gene which codes for maltooligosyl-trehalose trehalohydrolase or the catalytic properties of the gene products can be reduced or eliminated. The two measures are optionally combined.

The gene expression can be reduced by suitable culturing or by genetic modification (mutation) of the signal structures of gene expression. Signal structures of gene expression are, for example, repressor genes, activator genes, operators, promoters, attenuators, ribosome binding sites, the start codon and terminators. The expert can find information on this e.g. in the patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1996)) and in known textbooks of genetics and molecular biology, such as e.g. the textbook by Knippers (“Molekulare Genetik”, 6th edition, Georg Thieme Verlag, Stuttgart, Germany, 1995) or that by Winnacker (“Gene und Klone”, VCH Verlagsgesellschaft, Weinheim, Germany, 1990).

Mutations which lead to a change or reduction in the catalytic properties of enzyme proteins are known from the prior art; examples which may be mentioned are the works by Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation und Struktur des Enzyms”, Reports from the Jülich Research Centre, Jül1-2906, ISSN09442952, Jülich, Germany, 1994). Summarizing descriptions can be found in known textbooks of genetics and molecular biology, such as e.g. that by Hagemann (“Allgemeine Genetik”, Gustav Fischer Verlag, Stuttgart, 1986).

Possible mutations are transitions, transversions, insertions and deletions. Depending on the effect of the amino acid exchange on the enzyme activity, “missense mutations” or “nonsense mutations” are referred to. Insertions or deletions of at least one base pair in a gene lead to “frame shift mutations”, as a consequence of which incorrect amino acids are incorporated or translation is interrupted prematurely. Deletions of several codons typically lead to a complete loss of the enzyme activity. Instructions on generation of such mutations are prior art and can be found in known textbooks of genetics and molecular biology, such as e.g. the textbook by Knippers (“Molekulare Genetik”, 6th edition, Georg Thieme Verlag, Stuttgart, Germany, 1995), that by Winnacker (“Gene und Kione”, VCH Verlagsgesellschaft, Weinheim, Germany, 1990) or that by Hagemann (“Allgemeine Genetik”, Gustav Fischer Verlag, Stuttgart, 1986).

A common method of mutating genes of C. glutamicum is the method of “gene disruption” and “gene replacement” described by Schwarzer and Pühler (Bio/Technology 9, 84-87 (1991)).

In the method of gene disruption a central part of the coding region of the gene of interest is cloned in a plasmid vector which can replicate in a host (typically E. coli), but not in C. glutamicum. Possible vectors are, for example, pSUP301 (Simon et al., Bio/Technology 1, 784-791 (1983)), pK18mob or pK19mob (Schäfer et al., Gene 145, 69-73 (1994)), pK18mobsacB or pK19mobsacB (Jager et al., Journal of Bacteriology 174: 5462-65 (1992)), pGEM-T (Promega Corporation, Madison, Wis., USA), pCR2.1-TOPO (Shuman (1994). Journal of Biological Chemistry 269:32678-84; U.S. Pat. No. 5,487,993), pCR®Blunt (Invitrogen, Groningen, Holland; Bernard et al., Journal of Molecular Biology, 234: 534-541 (1993)) or pEM1 (Schrumpf et al, 1991, Journal of Bacteriology 173:4510-4516). The plasmid vector which contains the central part of the coding region of the gene is then transferred into the desired strain of C. glutamicum by conjugation or transformation. The method of conjugation is described, for example, by Schäfer et al. (Applied and Environmental Microbiology 60, 756-759 (1994)). Methods for transformation are described, for example, by Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a “cross-over” event, the coding region of the gene in question is interrupted by the vector sequence and two incomplete alleles are obtained, one lacking the 3′ end and one lacking the 5′ end. This method has been used, for example, by Fitzpatrick et al. (Applied Microbiology and Biotechnology 42, 575-580 (1994)) to eliminate the recA gene of C. glutamicum.

In the method of “gene replacement”, a mutation, such as e.g. a deletion, insertion or base exchange, is established in vitro in the gene of interest. The allele prepared is in turn cloned in a vector which is not replicative for C. glutamicum and this is then transferred into the desired host of C. glutamicum by transformation or conjugation. After homologous recombination by means of a first “cross-over” event which effects integration and a suitable second “cross-over” event which effects excision in the target gene or in the target sequence, the incorporation of the mutation or of the allele is achieved. This method was used, for example, by Peters-Wendisch et al. (Microbiology 144, 915 -927 (1998)) to eliminate the pyc gene of C. glutamicum by a deletion.

A deletion, insertion or a base exchange can be incorporated in this manner into the gene which codes for trehalose phosphatase and/or the gene which codes for maltooligosyl-trehalose synthase and/or the gene which codes for maltooligosyl-trehalose trehalohydrolase.

In addition, it may be advantageous for the production of L-amino acids to enhance, in particular over-express, one or more enzymes of the particular biosynthesis pathway, of glycolysis, of anaplerosis, of the citric acid cycle, of the pentose phosphate cycle, of amino acid export and optionally regulatory proteins, in addition to the attenuation of the gene which codes for trehalose phosphatase and/or the gene which codes for maltooligosyl-trehalose synthase and/or the gene which codes for maltooligosyl-trehalose trehalohydrolase.

The term “enhancement” or “enhance” in this connection describes the increase in the intracellular activity of one or more enzymes or proteins in a microorganism which are coded by the corresponding DNA, for example by increasing the number of copies of the gene or genes, using a potent promoter or a gene which codes for a corresponding enzyme or protein with a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on that of the wild-type protein or the activity or concentration of the protein in the starting microorganism.

Thus, for the production of amino acids, in particular L-lysine or L-glutamic acid, in addition to the attenuation of the gene which codes for trehalose phosphatase and/or the gene which codes for maltooligosyl-trehalose synthase and/or the gene which codes for maltooligosyl-trehalose trehalohydrolase, one or more of the genes chosen from the group consisting of
the lysC gene which codes for a feed-back resistant aspartate kinase (Accession No.P26512, EP-B-0387527; EP-A-0699759; WO 00/63388),
the dapA gene which codes for dihydrodipicolinate synthase (EP-B 0 197 335),
the gap gene which codes for glyceraldehyde 3-phosphate dehydrogenase (Eikmanns (1992). Journal of Bacteriology 174:6076-6086),
at the same time the pyc gene which codes for pyruvate carboxylase (DE-A-198 31 609),
the mqo gene which codes for malate:quinone oxidoreductase (Molenaar et al., European Journal of Biochemistry 254, 395-403 (1998)), (attenuation or enhancement???)
the zwf gene which codes for glucose 6-phosphate dehydrogenase (JP-A-09224661),
at the same time the lysE gene which codes for lysine export (DE-A-195 48 222),
the zwa1 gene which codes for the Zwa1 protein (DE: 19959328.0, DSM 13115)
the tpi gene which codes for triose phosphate isomerase (Eikmanns (1992), Journal of Bacteriology 174:6076-6086), and
the pgk gene which codes for 3-phosphoglycerate kinase (Eikmanns (1992), Journal of Bacteriology 174:6076-6086),
   can be enhanced, in particular over-expressed.

It may furthermore be advantageous for the production of amino acids, in particular L-lysine or L-glutamic acid, in addition to the attenuation of the gene which codes for trehalose phosphatase and/or the gene which codes for maltooligosyl-trehalose synthase and/or the gene which codes for maltooligosyl-trehalose trehalohydrolase, at the same time for one or more of the genes chosen from the group consisting of
the pck gene which codes for phosphoenol pyruvate carboxykinase (DE 199 50 409.1, DSM 13047),
the pgi gene which codes for glucose 6-phosphate isomerase (U.S. Ser. No. 09/396,478, DSM 12969),
the poxB gene which codes for pyruvate oxidase (DE: 1995 1975.7, DSM 13114),

the zwa2 gene which codes for the Zwa2 protein (DE: 19959327.2, DSM 13113),
the horn gene which codes for homoserine dehydrogenase (EP-A-0131171) and
the thrB gene which codes for homoserine kinase (Peoples, O. W., et al., Molecular Microbiology 2 (1988): 63-72)
   to be attenuated, in particular for the expression thereof to be reduced.

Finally, it may be advantageous for the production of amino acids, in addition to the attenuation of the gene which codes for trehalose phosphatase and/or the gene which codes for maltooligosyl-trehalose synthase and/or the gene which codes for maltooligosyl-trehalose trehalohydrolase, to eliminate undesirable side reactions (Nakayama: “Breeding of Amino Acid Producing Micro-organisms”, in: Overproduction of Microbial Products, Krumphanzl, Sikyta, Vanek (eds.), Academic Press, London, UK, 1982).

The invention also provides the microorganisms prepared according to the invention, and these can be cultured continuously or discontinuously in the batch process (batch culture) or in the fed batch (feed process) or repeated fed batch process (repetitive feed process) for the purpose of production of L-amino acids. A summary of known culture methods is described in the textbook by Chmiel (Bioprozesstechnik 1. Einführung in die Bioverfahrenstechnik (Gustav Fischer Verlag, Stuttgart, 1991)) or in the textbook by Storhas (Bioreaktoren und periphere Einrichtungen (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981).

Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substances can be used individually or as a mixture.

Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture.

Phosphoric acid, potassium dihydrogen phosphate or dipotassium hydrogen phosphate or the corresponding sodium-containing salts can be used as the source of phosphorus. The culture medium must furthermore comprise salts of metals, such as e. g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH of the culture. Antifoams, such as e.g. fatty acid polyglycol esters, can be employed to control the development of foam. Suitable substances having a selective action, such as e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 20° C. to 45° C., and preferably 25° C. to 40° C. Culturing is continued until a maximum of the desired product has formed. This target is usually reached within 10 hours to 160 hours.

Methods for the determination of L-amino acids are known from the prior art. The analysis can thus be carried out as described by Spackman et al. (Analytical Chemistry, 30, (1958), 1190) by anion exchange chromatography with subsequent ninhydrin derivatization, or it can be carried out by reversed phase HPLC, for example as described by Lindroth et al. (Analytical Chemistry (1979) 51: 1167-1174).

### EXAMPLES

Having generally described this invention, a further understanding can be obtained by reference to certain specific examples which are provided herein for purposes of illustration only and are not intended to be limiting unless otherwise specified.

### Example 1

### Preparation of Integration Vectors for Integration

### Mutagenesis of the otsB, treY and treZ Genes

From the strain ATCC 13032, chromosomal DNA is isolated by the method of Eikmanns et al. (Microbiology 140: 1817-1828 (1994)).

On the basis of the sequence of the otsB, treY and treZ genes known for C. glutamicum (WO 01/00843), the following oligonucleotides are chosen for the polymerase chain reaction:

otsB-int1:
5′ GTC CGA TTT TGA TGG AAC C 3′
otsB-int2:
5′ GGA GCT GAT GGA GTA TTC G 3′
treY-int1:
5′ TTT TCC GTG AAT ACG TTG G 3′
treY-int2:
5′ GCG ACT AAT TCG ATG ATG G 3′
treZ-int1:
5′ TGG TTC GAA GAT TTT CAC G 3′
treZ-int2:
5′ GGC GAG CTG TAG ATA ATG G 3′

The primers shown are synthesized by MWG Biotech (Ebersberg, Germany) and the PCR reaction is carried out by the standard PCR method of Innis et al. (PCR Protocols. A Guide to Methods and Applications, 1990, Academic Press) with the Taq-polymerase from Boehringer Mannheim (Germany, Product Description Taq DNA polymerase, Product No. 1 146 165). With the aid of the polymerase chain reaction, the primers allow amplification of an internal fragment of the otsB gene 463 bp in size, an internal fragment of the treY gene 530 bp in size and an internal fragment of the treZ gene 530 bp in size. The products amplified in this way are tested electrophoretically in a 0.8% agarose gel.

The amplified DNA fragments are ligated with the TOPO TA Cloning Kit from Invitrogen Corporation (Carlsbad, Calif., USA; Catalogue Number K4500-01) in each case in the vector pCR2.1-TOPO (Mead at al. (1991) Bio/Technology 9:657-663).

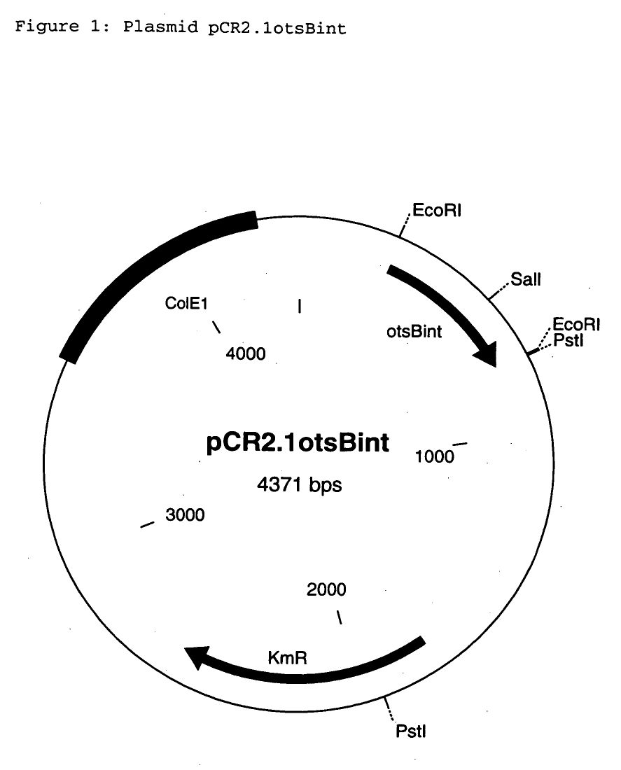

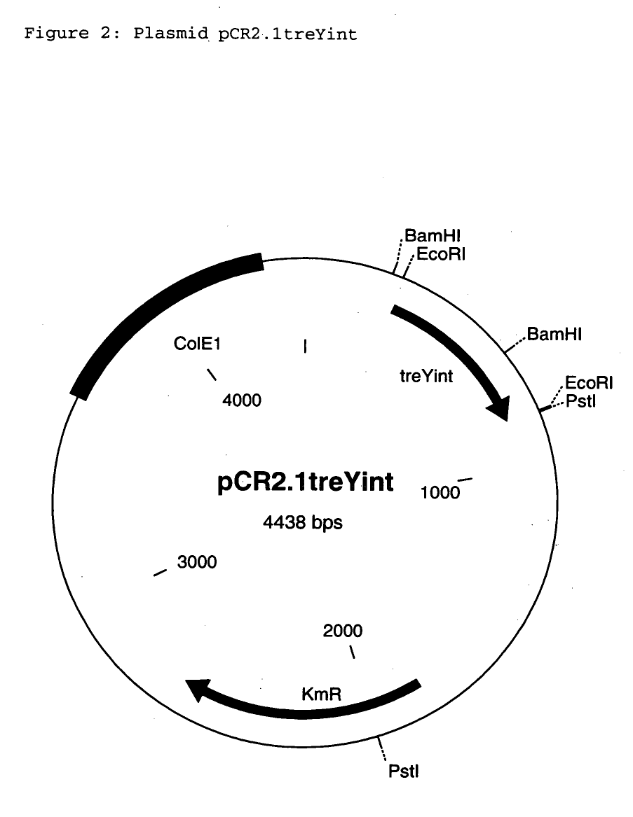

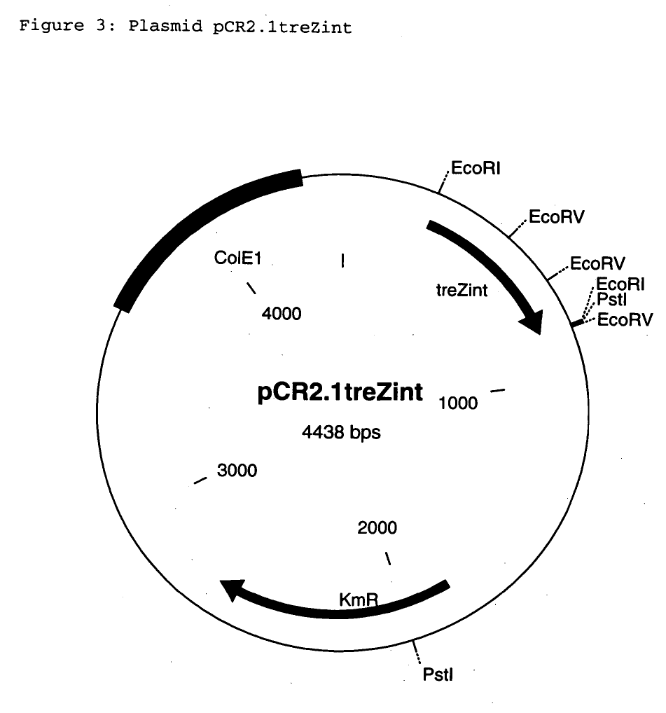

The E. coli strain TOP10 is then electroporated with the ligation batches (Hanahan, In: DNA Cloning. A Practical Approach. Vol. I, IRL-Press, Oxford, Washington D.C., USA, 1985). Selection for plasmid-carrying cells is made by plating out the transformation batch on LB agar (Sambrook et al., Molecular Cloning: A Laboratory Manual. 2^{nd} Ed., Cold Spring Harbor Laboratory Press, Cold Spring Harbor, N.Y., 1989), which had been supplemented with 50 mg/l kanamycin. Plasmid DNA is isolated from in each case one transformant with the aid of the QIAprep Spin Miniprep Kit from Qiagen and checked by restriction with the restriction enzyme EcoRI and subsequent agarose gel electrophoresis (0.8%). The plasmids are called pCR2.1otsBint, pCR2.1treYint and pCR2.1treZint and are shown in FIG. 1, FIG. 2 and FIG. 3.

The following microorganisms are deposited as a pure culture on 24 Apr. 2001 at the Deutsche Sammlung für Mikroorganismen und Zellkulturen (DSMZ=German Collection of Microorganisms and Cell Cultures, Braunschweig, Germany) in accordance with the Budapest Treaty:
Escherichia coli Top 10/pCR2.1otsBint as DSM 14259,
Escherichia coli Top 10/pCR2.1treYint as DSM 14260,
Escherichia coli Top 10/pCR2.1treZint as DSM 14261.

### Example 2

### Integration Mutagenesis of the otsB Gene in the Strain DSM 5715

The vector pCR2.1otsBint mentioned in example 1 is electroporated by the electroporation method of Tauch et al.(FEMS Microbiological Letters, 123:343-347 (1994)) in Corynebacterium glutamicum DSM 5715. The strain DSM 5715 is an AEC-resistant lysine producer. The vector pCR2.1otsBint cannot replicate independently in DSM5715 and is retained in the cell only if it has integrated into the chromosome of DSM 5715. Selection of clones with pCR2.1otsBint integrated into the chromosome is carried out by plating out the electroporation batch on LB agar (Sambrook et al., Molecular cloning: A Laboratory Manual. 2^{nd} Ed., Cold Spring Harbor Laboratory Press, Cold Spring Harbor, N.Y.), which had been supplemented with 15 mg/l kanamycin.

For detection of the integration, the otsBint fragment is labelled with the Dig hybridization kit from Boehringer by the method of “The DIG System Users Guide for Filter Hybridization” of Boehringer Mannheim GmbH (Mannheim, Germany, 1993). Chromosomal DNA of a potential integrant is isolated by the method of Eikmanns et al. (Microbiology 140: 1817-1828 (1994)) and in each case cleaved with the restriction enzymes EcoRI, SalI and PstI. The fragments formed are separated by means of agarose gel electrophoresis and hybridized at 68° C. with the Dig hybridization kit from Boehringer. The plasmid pCR2.1otsBint mentioned in example 3 has been inserted into the chromosome of DSM5715 within the chromosomal otsB gene. The strain is called DSM5715::pCR2.1otsBint.

### Example 3

### Integration Mutagenesis of the treY Gene in the Strain DSM 5715

The vector pCR2.1treYint mentioned in example 1 is electroporated by the electroporation method of Tauch et al.(FEMS Microbiological Letters, 123:343-347 (1994)) in Corynebacterium glutamicum DSM 5715. The strain DSM 5715 is an AEC-resistant lysine producer. The vector pCR2.1treYint cannot replicate independently in DSM5715 and is retained in the cell only if it has integrated into the chromosome of DSM 5715. Selection of clones with pCR2.1treYint integrated into the chromosome is carried out by plating out the electroporation batch on LB agar (Sambrook et al., Molecular cloning: A Laboratory Manual. 2^{nd} Ed., Cold Spring Harbor Laboratory Press, Cold Spring Harbor, N.Y.), which had been supplemented with 15 mg/l kanamycin.

For detection of the integration, the treYint fragment is labelled with the Dig hybridization kit from Boehringer by the method of “The DIG System Users Guide for Filter Hybridization” of Boehringer Mannheim GmbH (Mannheim, Gerrnany, 1993). Chromosomal DNA of a potential integrant is isolated by the method of Eikmanns et al. (Microbiology 140: 1817-1828 (1994)) and in each case cleaved with the restriction enzymes EcoRI, BamHI and PstI. The fragments formed are separated by means of agarose gel electrophoresis and hybridized at 68° C. with the Dig hybridization kit from Boehringer. The plasmid pCR2.1treYint mentioned in example 3 has been inserted into the chromosome of DSM5715 within the chromosomal treY gene. The strain is called DSM5715::pCR2.1treYint.

### Example 4

### Integration Mutagenesis of the treZ Gene in the Strain DSM 5715

The vector pCR2.1treZint mentioned in example 1 is electroporated by the electroporation method of Tauch et al.(FEMS Microbiological Letters, 123:343-347 (1994)) in Corynebacterium glutamicum DSM 5715. The strain DSM 5715 is an AEC-resistant lysine producer. The vector pCR2.1treZint cannot replicate independently in DSM5715 and is retained in the cell only if it has integrated into the chromosome of DSM 5715. Selection of clones with pCR2.1treZint integrated into the chromosome is carried out by plating out the electroporation batch on LB agar (Sambrook et al., Molecular cloning: A Laboratory Manual. 2^{nd} Ed., Cold Spring Harbor Laboratory Press, Cold Spring Harbor, N.Y.), which had been suppimented with 15 mg/l kanamycin.

For detection of the integration, the treZint fragment is labelled with the Dig hybridization kit from Boehringer by the method of “The DIG System Users Guide for Filter Hybridization” of Boehringer Mannheim GmbH (Mannheim, Germany, 1993). Chromosomal DNA of a potential integrant is isolated by the method of Eikmanns et al. (Microbiology 140: 1817-1828 (1994)) and in each case cleaved with the restriction enzymes EcoRI, EcoRV and PstI. The fragments formed are separated by means of agarose gel electrophoresis and hybridized at 68° C. with the Dig hybridization kit from Boehringer. The plasmid pCR2.1treZint mentioned in example 3 has been inserted into the chromosome of DSM5715 within the chromosomal treZ gene. The strain is called DSM5715::pCR2.1treZint.

### Example 5

### Preparation of Lysine

The C. glutamicum strains DSM5715::pCR2.1otsbint, DSM5715::pCR2.1treYint and DSM5715::pCR2.1treZint obtained in example 2, example 3 and example 4 are cultured in a nutrient medium suitable for the production of lysine and the lysine content in the culture supernatant is determined.

For this, the strains are first incubated on an agar plate with the corresponding antibiotic (brain-heart agar with kanamycin (25 mg/l) for 24 hours at 33° C. Starting from this agar plate culture, in each case a preculture is seeded (10 ml medium in a 100 ml conical flask). The complete medium CgIII is used as the medium for the preculture.

Medium Cg III

| | | |
|---|---|---|
| NaCl | 2.5 | g/l |
| Bacto-Peptone | 10 | g/l |
| Bacto-Yeast extract | 10 | g/l |
| Glucose (autoclaved separately) | 2% | (w/v) |
| The pH is brought to pH 7.4 | | |

Kanamycin (25 mg/l) is added to this. The precultures are incubated for 16 hours at 33° C. at 240 rpm on a shaking machine. In each case a main culture is seeded from these precultures such that the initial OD (660 nm) of the main cultures is 0.1. Medium MM is used for the main culture.

Medium MM

| | | |
|---|---|---|
| CSL (corn steep liquor) | 5 | g/l |
| MOPS (morpholinopropanesulfonic acid) | 20 | g/l |
| Glucose (autoclaved separately) | 50 | g/l |
| Salts: | | |
| (NH₄)₂SO₄ | 25 | g/l |
| KH₂PO₄ | 0.1 | g/l |
| MgSO₄ * 7H₂O | 1.0 | g/l |
| CaCl₂ * 2H₂O | 10 | mg/l |
| FeSO₄ * 7H₂O | 10 | mg/l |
| MnSO₄ * H₂O | 5.0 | mg/l |
| Biotin (sterile-filtered) | 0.3 | mg/l |
| Thiamine * HCl (sterile-filtered) | 0.2 | mg/l |
| Leucine (sterile-filtered) | 0.1 | g/l |
| CaCO₃ | 25 | g/l |

The CSL, MOPS and the salt solution are brought to pH 7 with aqueous ammonia and autoclaved. The sterile substrate and vitamin solutions are then added, and the CaCO₃ autoclaved in the dry state is added.

Culturing is carried out in a 10 ml volume in 100 ml conical flasks with baffles. Kanamycin (25 mg/l) was added. Culturing is carried out at 33° C. and 80% atmospheric humidity.

After 72 hours, the OD is determined at a measurement wavelength of 660 nm with a Biomek 1000 (Beckmann Instruments GmbH, Munich). The amount of lysine formed is in each case determined with an amino acid analyzer from Eppendorf-BioTronik (Hamburg, Germany) by ion exchange chromatography and post-column derivatization with ninhydrin detection.

The result of the experiment is shown in table 1.

TABLE 1

| Strain | OD | Lysine HCl |
|---|---|---|
| DSM5715 | 7.3 | 12.48 |
| DSM5715::pCR2.1otsBint | 7.5 | 13.45 |
| DSM5715::pCR2.1treYint | 7.5 | 13.13 |
| DSM5715::pCR2.1treZint | 8.1 | 13.84 |

Obviously, numerous modifications and variations of the present invention are possible in light of the above teachings. It is therefore to be understood that within the scope of the appended claims, the invention may be practiced otherwise than as specifically described herein.

All of the publications cited above are incorporated herein by reference.

1-21. (canceled)

22. A process for the fermentative preparation of L-lysine, comprising:
a) fermenting in a medium a Corynebacterium glutamicum which produces L-lysine and in which at least one gene native to the Corynebacterium glutamicum and selected from the group consisting of a gene coding for trehalose phosphatase, a gene coding for maltooliogosyl-trehalose synthase and a gene coding for maltooliogosyl-trehalose trehalohydrolase is eliminated,
b) concentrating the L-lysine in the medium or in the cells of the Corynebacterium glutamicum and
c) isolating the L-lysine optionally with constituents of the fermentation medium and/or biomass remaining in the portions or in their total amounts in the end product,
wherein the elimination is achieved by a method of mutagenesis selected from the group consisting of insertion mutagenesis with insertion of at least one nucleotide, deletion mutagenesis with deletion of at least one nucleotide and transition or transversion mutagenesis with incorporation of a stop codon.

23. The process of claim 22, wherein the trehalose phosphatase comprises the amino acid sequence of SEQ ID NO: 2, wherein the maltooligosyl-trehalose synthase comprises the amino acid sequence of SEQ ID NO: 4 and wherein the maltooligosyl-trehalose trehalohydrolase comprises the amino acid sequence of SEQ ID NO: 6.

24. The process of claim 22, wherein the trehalose phosphatase gene comprises the nucleotide sequence of nucleotides 601 to 1368 of SEQ ID NO: 1, wherein the maltooligosyl-trehalose synthase gene comprises the nucleotide sequence of nucleotides 601 to 3033 of SEQ ID NO: 3 and wherein the maltooligosyl-trehalose trehalohydrolase gene comprises the nucleotide sequence of nucleotides 601 to 2430 of SEQ ID NO: 5.

25. The process of claim 22, wherein the trehalose phosphatase gene comprises the nucleotide sequence of SEQ ID NO: 1, wherein the maltooligosyl-trehalose synthase gene comprises the nucleotide sequence of SEQ ID NO: 3 and wherein the maltooligosyl-trehalose trehalohydrolase gene comprises the nucleotide sequence of SEQ ID NO: 5.

26. The process of claim 22, wherein one or more genes native to Corynebacterium glutamicum and selected from the group consisting of
a gene coding for aspartate kinase,
a gene coding for dihydrodipicolinate synthase,
a gene coding for glyceraldehydes 3-phosphate dehydrogenase,
a gene coding for pyruvate carboxylase,
a gene coding for malate: quinine oxidoreductase,
a gene coding for glucose 6-phosphate dehydrogenase,
a gene coding for a protein that exports lysine,
a gene coding for the Zwa1 protein,
a gene coding for triose phosphate isomerase, and
a gene coding for 3-phosphoglycerate kinase,
is or are over-expressed by increasing the copy number or by operably linking a promoter to the gene.

27. The process of claim 22, wherein one or more genes native to Corynebacterium glutamicum and selected from the group consisting of
a gene coding for phosphoenol pyruvate carboxylkinase,
a gene coding for glucose 6-phosphate isomerase,
a gene coding for for pyruvate oxidase,
a gene coding for the Zwa2 protein,
a gene coding for homoserine dehydrogenase
a gene coding for homoserine kinase,
is or are eliminated by a method of mutagenesis selected from the group consisting of insertion mutagenesis with insertion of at least one nucleotide, deletion mutagenesis with deletion of at least one nucleotide and transition or transversion mutagenesis with incorporation of a stop codon.

28. The process of claim 22, wherein for insertion mutagenesis of the gene coding for trehalose phosphatase plasmid pCR2.1otsBint deposited under DSM 14259 is used.

29. The process of claim 22, wherein for insertion mutagenesis of the gene coding for maltooligosyl-trehalose synthase plasmid pCR2.1treYint deposited under DSM 14260 is used.

30. The process of claim 22, wherein for insertion mutagenesis of the gene coding for maltooligosyl-trehalose synthase plasmid pCR2.1treZint deposited under DSM 14261 is used.

31. The process of claim 22, wherein at least the gene coding for trehalose phosphatase is eliminated.

32. The process of claim 22, wherein at least the gene coding for maltooliogosyl-trehalose synthase is eliminated.

33. The process of claim 22, wherein at least the gene coding for maltooliogosyl-trehalose trehalohydrolase is eliminated.

34. The process of claim 22, wherein the elimination is achieved by insertion mutagenesis with insertion of at least one nucleotide.

35. The process of claim 22, wherein the elimination is achieved by deletion mutagenesis with deletion of at least one nucleotide and transition.

36. The process of claim 22, wherein the elimination is achieved by transversion mutagenesis with incorporation of a stop codon.

37. The process of claim 22, wherein the elimination is achieved by transition mutagenesis with incorporation of a stop codon.


SEQUENCE LISTING (12 sequences)

SEQ ID NO: 1 - DNA, 1971 bp, Corynebacterium glutamicum
  CDS (601)..(1368)
cgtcaacgtgtgggctaatagtttcctggattgtttggcacagtcgggagaaaactcatgaaccgcgcacgaatcgcgaccataggcgttcttccgcttgctttactgctggcgtcctgtggttcagacaccgtggaaatgacagattccacctggttggtgaccaatatttacaccgatccagatgagtcgaattcgatcagtaatcttgtcatttcccagcccagcttagattttggcaattcttccctgtctggtttcactggctgtgtgccttttacggggcgtgcggaattcttccaaaatggtgagcaaagctctgttctggatgccgattatgtgaccttgtcttccctggatttcgataaacttcccgatgattgccaaggacaagaactcaaagttcataacgagctggttgatcttctgcctggttcttttgaaatctccaggacttctggttcagaaatcttgctgactagcgatgtcgatgaactcgatcggccagcaatccgcttggtgtcctggatcgcgccgacatcttaaggtgccagggctttaaagtgccaggggttctgtgggatccgtacactggttcccatgactttgactattgaggaaatcgccaagaccaaaaagcttttggttgtgtccgattttgatggaaccatcgcaggatttagcaaggacgcttacaacgttcctatcaaccagaaatccctcaaggcggtaaaagacctctcccaacaagcagacactgatgttgtcattttgtcgggacgtcacctggagggattgaagacggttcttgatcttggtcagtacgacatcaccatggtgggttcacacggttctgaggattcctcccgcccgcgtaccctcactcctgaagaggtagctcgcctcgccaagattgaagcagatctggaaaagatcgtcgacggcatcgaaggcgcattcgtggagatcaagcctttccaccgcgtgctgcacttcatccgtgtttccgacaaggacaaagtccaaggaatcctcgcccaagcagcacacgtagactcttccggcctgaaggttactaacggcaagagcatcatcgaatactccatcagctccaccaccaagggcacctggctgaaggaatacgttgaccgcaccgagcccactggtgtgattttcctcggcgatgacaccaccgatgagcacggtttcaaagctttagaaaacgatgatcgtgccctaaccgtcaaggttggcgaaggagacactgcagccaaaacccgcgtcgacgatgttgataatgtgggaattttcctagagaaactcgcctaccaccgcatgcagtatgcggaaagcgtgcgattggggatttaagagagcctaaacgcacgaaaagtgccacaagcctagattggcgcaaccgtggaacctgggtggaaggaagtttccaattccacttctggttccacgtattctttgtcaatcattttcaacagtgtttcgccggctttgaaccctttgagtttgttgggttggatgacggtggtgagatcccgtgcgagtgccatgtgggtgccatcgaaaccagtgagggatagatctgcaggcgctgatttacctacgcttttaaggtattccagaacgccgaatgccagtgcatcgacggtacagagtactgcggtgaggtctgggtgtgtttctagaagttctttggccacttcgaagttgtgttggcggttgttgatccagcattccatgatcggcacggtgccgggatcgattcccgcttcgataaagacttccatggcacccctgactcggtcgcgttgtacctggtattgggcgttttcgaggcgctcgcgggtgacttcgccgtcgttgtttgcgcggtctaggcggatggacaggatgccgattttgcggtggccggcgtcgattagcgcttgagctgcaggggcgatggctttgcggtta

SEQ ID NO: 2 - protein, 256 aa, Corynebacterium glutamicum
MTLTIEEIAKTKKLLVVSDFDGTIAGFSKDAYNVPINQKSLKAVKDLSQQADTDVVILSGRHLEGLKTVLDLGQYDITMVGSHGSEDSSRPRTLTPEEVARLAKIEADLEKIVDGIEGAFVEIKPFHRVLHFIRVSDKDKVQGILAQAAHVDSSGLKVTNGKSIIEYSISSTTKGTWLKEYVDRTEPTGVIFLGDDTTDEHGFKALENDDRALTVKVGEGDTAAKTRVDDVDNVGIFLEKLAYHRMQYAESVRLGI

SEQ ID NO: 3 - DNA, 3636 bp, Corynebacterium glutamicum
  CDS (601)..(3033)
ataacccagcaatgctgaacctttcccacgatctgtgtatggggttgcacagcgggaaagtcacactcagttaccgggctctcgctgaagtggggctgaccaccaggcaagcgtgggatcaatcagccaccaatttattaaagtgtgctactactcctgagggtattcgatttgatcttcgggacgcagaaacgaccacaaccatcgcctcgtctgcattagaaatcagagttcccggcgcgccaattactgcatggttggcgcatcctcaaacctttacagtgttgaatcgtcatcttgaattacgactgggaccagcaccgctgtatcttgctccgaattcacacacactcatcgctattcctgctggtgatccagggatctttgaatttgaacggtgggcgcgaagcttgctggaagtgggtggtgtggagggtatcgtcgataagcttcttgtttataggcacggctttccttgcccttaccagccagcctttgtagcacttgctgcgtaaatctttttcccacgccgggaatgcgtgaacactaagatcgaggacgtaccgcacgattttgcctaacttttaagggtgtttcatcatggcacgtccaatttccgcaacgtacaggcttcaaatgcgaggacctcaagcagatagcgccgggcgttcatttggttttgcgcaggccaaagcccagcttccctatctgaagaagctaggcatcagccacctgtacctctcccctatttttacggccatgccagattccaatcatggctacgatgtcattgatcccaccaccatcaatgaagagctcggtggcatggagggtcttcgagatcttgccgcagctacacacgagttgggcatgggcatcatcattgatattgttcccaaccatttaggtgttgccgttccacatttgaatccttggtggtgggatgttctaaaaaacggcaaagattccgcttttgagttctatttcgatattgactggcacgaagacaacggttctggtggcaagctgggcatgccgattctgggtgctgaaggcgatgaagacaagctggaattcgcggagcttgatggagagaaagtgctcaaatattttgaccacctcttcccaatcgcgcctggtaccgaagaagggacaccgcaagaagtctacaagcgccagcattaccgcctgcagttctggcgcgatggcgtgatcaacttccgtcgcttcttttccgtgaatacgttggctggcatcaggcaagaagatcccttagtgtttgaacatactcatcgtctgctgcgcgaattggtggcggaagacctcattgacggcgtgcgcgtcgatcaccccgacgggctttccgatccttttggatatctgcacagactccgcgacctcattggacctgaccgctggctgatcatcgaaaagatcttgagcgttgatgaaccactcgatccccgcctggccgttgatggcaccactggctacgacgccctccgtgaactcgacggcgtgtttatctcccgagaatctgaggacaaattctccatgctggcgctgacccacagtggatccacctgggatgaacgcgccctcaaatccacggaggaaagcctcaaacgagtcgtcgcccaacaagaactcgcagccgaaatcttaaggctcgcccgcgccatgcgccgcgataacttctccaccgcaggcaccaacgtcaccgaagacaaacttagcgaaaccatcatcgaattagtcgccgccatgcccgtctaccgcgccgactacatctccctctcacgcaccaccgccaccgtcatcgcggagatgtccaaacgcttcccctcccggcgtgacgcactcgacctcatcgcggccgccctacttggcaatggcgaggccaaaatccgcttcgctcaagtctgcggcgccgtcatggctaaaggtgtggaagacaccaccttctaccgcgcatctaggctcgttgcattgcaagaagtcggtggcgcgccggggagattcggcgtctccgctgcagaattccacttgctgcaggaagaacgcagcctgctgtggccacgcaccatgaccaccttgtccacgcatgacaccaaacgtggcgaagatacccgcgcccgcatcatctccctgtctgaagtccccgatatgtactccgagctggtcaatcgtgttttcgcggtgctccccgcgccagacggcgcaacgggcagtttcctcctacaaaacctgctgggcgtatggcccgccgacggcgtgatcaccgatgcgctgcgcgatcgattcagggaatacgccctaaaagctatccgcgaagcatccacaaaaaccacgtgggtggaccccaacgagtccttcgaggctgcggtctgcgattgggtggaagcgcttttcgacggaccctccacctcactaatcaccgaatttgtctcccacatcaaccgtggctctgtgcaaatctccttaggcaggaaactgctgcaaatggtgggcgctggaatccccgacacttaccaaggaactgagtttttagaagactccctggtagatcccgataaccgacgctttgttgattacaccgccagagaacaagtcctggagcgcctgcaaacctgggattggacgcaggttaattcggtagaagacttggtggataacgccgacatcgccaaaatggccgtggtccataaatccctcgagttgcgtgctgaatttcgtgcaagctttgttggtggagatcatcaggcagtatttggcgaaggtcgcgcagaatcccacatcatgggcatcgcccgcggtacagaccgaaaccacctcaacatcattgctcttgctacccgtcgaccactgatcttggaagaccgtggcggatggtatgacaccaccgtcacgcttcctggtggacaatgggaagacaggctcaccgggcaacgcttcagtggtgttgtcccagccaccgatttgttctcacatctacccgtatctttgttggttttagtacccgatagtgagttttgatccctgcacaggaaagttagcggcgctactatgaacgatcgatatgtctgacaacactctctcccaatttggcagttactaccacgaattccgacgtgcccatcccatggccgacgtcgaattcctcctagcaattgaagaattacttacggacggtggtgtcaccttcgatcgcgtcaccacacgcatcaaagaatggtcaagcctgaaagccaaggctcgcaagcgtcgcaacgatggctcgttgatctaccctgatccgcgcaaagacatccacgacatgatcggtgttcggatcaccacgtaccactccacggaaatacccgtggccctaaaagtgctccaagactccttcatcgtccacaaatccgtagacaaagccgctgaaactcgcatctcaggcggctttggttacggctcccaccacctgattctggaagtcgatgacacctccgatgacctccaggactacaaaggcctcgtctttgaagttcaggtgcgcaccgtgctgcaacacgcctgggcagagttcgaacacgatatccgctataaacgcgccgatgtgtccaacccagaagacttcagcgcagaagtagaccg

SEQ ID NO: 4 - protein, 811 aa, Corynebacterium glutamicum
MARPISATYRLQMRGPQADSAGRSFGFAQAKAQLPYLKKLGISHLYLSPIFTAMPDSNHGYDVIDPTTINEELGGMEGLRDLAAATHELGMGIIIDIVPNHLGVAVPHLNPWWWDVLKNGKDSAFEFYFDIDWHEDNGSGGKLGMPILGAEGDEDKLEFAELDGEKVLKYFDHLFPIAPGTEEGTPQEVYKRQHYRLQFWRDGVINFRRFFSVNTLAGIRQEDPLVFEHTHRLLRELVAEDLIDGVRVDHPDGLSDPFGYLHRLRDLIGPDRWLIIEKILSVDEPLDPRLAVDGTTGYDALRELDGVFISRESEDKFSMLALTHSGSTWDERALKSTEESLKRVVAQQELAAEILRLARAMRRDNFSTAGTNVTEDKLSETIIELVAAMPVYRADYISLSRTTATVIAEMSKRFPSRRDALDLIAAALLGNGEAKIRFAQVCGAVMAKGVEDTTFYRASRLVALQEVGGAPGRFGVSAAEFHLLQEERSLLWPRTMTTLSTHDTKRGEDTRARIISLSEVPDMYSELVNRVFAVLPAPDGATGSFLLQNLLGVWPADGVITDALRDRFREYALKAIREASTKTTWVDPNESFEAAVCDWVEALFDGPSTSLITEFVSHINRGSVQISLGRKLLQMVGAGIPDTYQGTEFLEDSLVDPDNRRFVDYTAREQVLERLQTWDWTQVNSVEDLVDNADIAKMAVVHKSLELRAEFRASFVGGDHQAVFGEGRAESHIMGIARGTDRNHLNIIALATRRPLILEDRGGWYDTTVTLPGGQWEDRLTGQRFSGVVPATDLFSHLPVSLLVLVPDSEF

SEQ ID NO: 5 - DNA, 3033 bp, Corynebacterium glutamicum
  CDS (601)..(2430)
ctagtcactcggggattccacccacatgtgcccggattctttcaattcaatttctgcacccatcatggatgacaattgagattccaccgcagcctgttcaccaggcaataaagccaaggtgtatgtgacttctgcaccatattcagtgtcagtaatgatgatgcccatgccgcgcagattcgcttcaatgcgccccgcatcagaatgcggaaggtcaatcttgaaaatctcccgaacagagcgggtgacttgcaaaacctcaggtagaagctccgtcaccgcgttggtgtaggcattaactaatccgccagtgcccagttttacgccaccgaaataacgcaccaccacagcggcaatatctttcattcccgagccacgcaacgcctctagcatgggttttcccgcggtaccggaaggttcgccatcatcagaggaacgctccacatcatttgagccatccacatggaaaataaaggcactgcaatgatgacgcgcatccggatacagctccttgatggagtgaataaattcgcgagcctgctcctgatcttgcacacgcgtgatataggtcagaaatcgcgagcgcttgatctctagttcatgctcaaagacttgaccggcctgagggagttggtattgcgtgagatgtgccatagcatctcacatcttagctcgccaaccggcagcattttcactagcctggtggccatgttgacctcgcaaagcttttcagtgtgggctccacttccccacgatgtacatctgatcctcaacggcgaaaccctccccatgcacaaaacggagggcagctggtggcgcgccgagatcgcgcccaaggccggcgatcgttacggtttttcgcttttcgacggctcctcctggtcaaaaaccctccccgatccccgctccacatctcaaccagacggggttcatggtttaagtgaagtctccgatgattcctatctgtggggtgaccagcagtggactggccgaattctccctggctcggtgttatatgagctgcatgtgggcacctttagtgaagatggaacgtttgagggagtcgtcgacaagcttccttatctgcgcgacctcggcgtgaccgccatcgaacttttacccgtgcagccctttggcggcaaccgcaattggggctacgacggggtgctgtggcacgccgtccatgcaggctacggcggtccggcgggcttgaaaaagcttatcgacgcctcccaccaggccggcatcgccgtctacttagacgtcgtgtacaaccacttcggccccgacggcaactacaacgggcaatttggcccctacacctctggcggcagcaccggctggggcgacgtggtcaacatcaacggccatgattcagatgaagtccgcaattatattctcgacgccgcacgccagtggttcgaagattttcacgttgatgggctccgcctcgatgcggtgcattctctcgatgatcgcggcgcctattccctacttgcgcagctgaccatggtggccgaggatgtctccgcacaaacaggcatcccacgctcattgattgcagaatctgaactcaatgaccccaagttcgttacctcccgcgaggccggcggttttggcctggatgcacagtgggttgacgatatccaccacgccctccatgccctcgtttctggcgaacgcaatggttattacagcgatttcggatctgtcgacacattagccaaaaccctgcgtgaagtatttgaacacaccggaaactactccacgtaccgcggacgcaaccacggccgccctgtgcaccccgatatcacccctgcctcgcgctttgtcacctacaccaccacccatgatcagaccggcaaccgcgcaatcggcgaccgtccttccacgactctcaccccggaacagcaggtgttgaaggcagccattatctacagctcgccgtataccccgatgttgtttatgggtgaagaattcggagccaccaccccattcgccttcttttgctcccacaccgaccccgagctcaaccgcctaacctccgagggccgcaaacgggaattcgcacgccttggctggaacgccgacgacatcccctcccccgagctggaatccaccttcacctcctccaaactcgattgggagttcactgcggagcagcgccgcatcaacgacgcttacaagcagctgttgcacctgcggcacaccttgggcttctcccaaccaaacttgctcacactcgaggttgagcacggcgagaactggctatcgatggccaatggtcgcggccgaattctggcgaatttctccgacgacaccatcaccgtcccgcttggcggcgagctgatttacagcttcacttcccccaccgtcaccgacacctccacaacccttcagccgtggggctttgcgatcctgacccgaaactagaaaaaggccacctcgattgaggtggccttcagctatgtttaggtcaagtattcgtactcaggggtgcccggctcgaggcgacgggaatcaattgccgattcctccatacgttccagcaaagaatccaatcctgatgcttcactcaagtgaatacccaccaacgcagtaccggtctcacggttgttgcgcttgaggtactcaaacagcgtgatgtcatcatccggtcccaggatatcttccaggaagtgacgcaactgaccaggcttttgcgggaagttcaccaagaagtagtgcttcaaaccgcggtgcaccaaggagcgctcagcgatttccgcataacgcagcacatcgttgttgccaccagagatgatgcacaccacgacagaaccaggtgcaaaggacatttccttcaacccagcgatagacagcgcgccagcaggctccgcgatgatgccttcgttttggtaaagatcgagcatctcagtacacacagcgccctcggtcgcgctcatcatgtgcacgcgaccctggttcttctccacgatggtgtagttgagatctccgacacgtttgactgctgcgccgtccacaaagggatca

SEQ ID NO: 6 - protein, 610 aa, Corynebacterium glutamicum
MLKDLTGLRELVLREMCHSISHLSSPTGSIFTSLVAMLTSQSFSVWAPLPHDVHLILNGETLPMHKTEGSWWRAEIAPKAGDRYGFSLFDGSSWSKTLPDPRSTSQPDGVHGLSEVSDDSYLWGDQQWTGRILPGSVLYELHVGTFSEDGTFEGVVDKLPYLRDLGVTAIELLPVQPFGGNRNWGYDGVLWHAVHAGYGGPAGLKKLIDASHQAGIAVYLDVVYNHFGPDGNYNGQFGPYTSGGSTGWGDVVNINGHDSDEVRNYILDAARQWFEDFHVDGLRLDAVHSLDDRGAYSLLAQLTMVAEDVSAQTGIPRSLIAESELNDPKFVTSREAGGFGLDAQWVDDIHHALHALVSGERNGYYSDFGSVDTLAKTLREVFEHTGNYSTYRGRNHGRPVHPDITPASRFVTYTTTHDQTGNRAIGDRPSTTLTPEQQVLKAAIIYSSPYTPMLFMGEEFGATTPFAFFCSHTDPELNRLTSEGRKREFARLGWNADDIPSPELESTFTSSKLDWEFTAEQRRINDAYKQLLHLRHTLGFSQPNLLTLEVEHGENWLSMANGRGRILANFSDDTITVPLGGELIYSFTSPTVTDTSTTLQPWGFAILTRN

SEQ ID NO: 7 - DNA, 19 bp, Corynebacterium glutamicum
gtccgattttgatggaacc

SEQ ID NO: 8 - DNA, 19 bp, Corynebacterium glutamicum
ggagctgatggagtattcg

SEQ ID NO: 9 - DNA, 19 bp, Corynebacterium glutamicum
ttttccgtgaatacgttgg

SEQ ID NO: 10 - DNA, 19 bp, Corynebacterium glutamicum
gcgactaattcgatgatgg

SEQ ID NO: 11 - DNA, 19 bp, Corynebacterium glutamicum
tggttcgaagattttcacg

SEQ ID NO: 12 - DNA, 19 bp, Corynebacterium glutamicum
ggcgagctgtagataatgg